(19) United States
(12) Patent Application Publication
(10) Pub. No.: US 2025/0295295 A1
(43) Pub. Date: Sep. 25, 2025

(54) MEDICAL OBSERVATION SYSTEM

(71) Applicant: Sony Olympus Medical Solutions Inc., Tokyo (JP)

(72) Inventor: Atsuomi OHNO, Tokyo (JP)

(73) Assignee: Sony Olympus Medical Solutions Inc., Tokyo (JP)

(21) Appl. No.: 19/077,064

(22) Filed: Mar. 12, 2025

(30) Foreign Application Priority Data
Mar. 19, 2024 (JP) 2024-044070

(51) Int. Cl. A61B 1/04; A61B 1/00

(52) U.S. Cl. CPC A61B 1/043; A61B 1/00009; A61B 1/042

(57) ABSTRACT

A medical observation system capable of suppressing an increase in size even in a case of configuring a three-dimensional endoscope includes a spectroscopic element that spectrally disperses light in a first optical path into light of a first and second wavelength band and spectrally disperses light in a second optical path different from the first optical path into light of a first and second wavelength band; a plurality of imaging elements image the light of the first and second wavelength band spectrally dispersed from the light in the first optical path and the light of the first and second wavelength bands spectrally dispersed from the light in the second optical path.

### CROSS-REFERENCE TO RELATED APPLICATION

This application claims the benefit of Japanese Priority Patent Application JP 2024-044070, filed on Mar. 19, 2024, the entire contents of which are incorporated herein by reference.

### TECHNICAL FIELD

The present disclosure relates to a medical observation system.

### BACKGROUND ART

Up to now, there has been known a medical observation system in which a fluorescent substance such as indocyanine green is administered into a living body, and an observation target is irradiated with excitation light that excites the fluorescent substance to fluorescently observe a lesion where the fluorescent substance is accumulated as a three-dimensional image.

### CITATION LIST

### Patent Literature

[PTL 1]
JP 2021-145873A

### SUMMARY

### Technical Problem

In a case where a three-dimensional endoscope capable of capturing both a three-dimensional image of white light and a three-dimensional image of fluorescence is configured, it is conceivable that one imaging element for white light, one imaging element for fluorescence, and one spectroscopic element (prism) that spectrally disperses white light and fluorescence are provided in the right eye, and further one imaging element for white light, one imaging element for fluorescence, and one spectroscopic element that spectrally disperses white light and fluorescence are also provided in the left eye. In this case, since a work space of an adhesive portion application jig is required between the two spectroscopic elements, it is necessary to arrange both spectroscopic elements separately, and there is a possibility that the endoscope becomes large in size.

The present disclosure provides a medical observation system capable of suppressing an increase in size even in a case where a three-dimensional endoscope is configured.

### Solution to Problem

According to the above present disclosure, there is provided a medical observation system including: a spectroscopic element that spectrally disperses light in a first optical path into light of a first wavelength band and light of a second wavelength band different from the first wavelength band, and spectrally disperses light in a second optical path different from the first optical path into light of the first wavelength band and light of the second wavelength band;
a first imaging element that images the light of the first wavelength band spectrally dispersed from the light in the first optical path;
a second imaging element that images the light of the second wavelength band spectrally dispersed from the light in the first optical path;
a third imaging element that images the light of the first wavelength band spectrally dispersed from the light in the second optical path; and
a fourth imaging element that images light of the second wavelength band spectrally dispersed from the light in the second optical path.

The spectroscopic dispersion of the light in the first optical path and the spectroscopic dispersion of the light in the second optical path may be performed by the same spectroscopic element.

A spectral surface that spectrally disperses the light in the first optical path and a spectral surface that spectrally disperses the light in the second optical path may be on the same plane of the same spectroscopic element.

The medical observation system may further include an image generation unit that generates a three-dimensional image of an output of the first imaging element, an output of the second imaging element, an output of the third imaging element, and an output of the fourth imaging element on the basis of the output of the first imaging element, the output of the second imaging element, the output of the third imaging element, and the output of the fourth imaging element.

The light in the first wavelength band may be white light, and the light in the second wavelength band may be first fluorescence.

Further, when a second fluorescence having a wavelength band different from that of the first fluorescence is imaged, the first imaging element and the third imaging element may image white light and the second fluorescence in a time division manner.

The spectroscopic element may have a first region through which light imaged by at least one of the first imaging element or the second imaging element passes and a second region through which light imaged by at least one of the third imaging element or the fourth imaging element passes, and
in the spectroscopic element, a hole may be provided in a region between the first region and the second region, and a convex portion of a base plate for fixing the spectroscopic element may be fitted into the hole.

In the spectroscopic element, two of the holes may be provided in the region between the first region and the second region, and two of the convex portions of the base plate for fixing the spectroscopic element may be fitted into the holes.

One of the two holes of the spectroscopic element may be a long hole.

The spectroscopic element may have a first region through which light imaged by at least one of the first imaging element or the second imaging element passes and a second region through which light imaged by at least one of the third imaging element or the fourth imaging element passes, and
in the spectroscopic element, a convex portion may be provided in a region between the first region and the second region, and the convex portion may be fitted into a hole of a base plate.

In the spectroscopic element, two convex portions may be provided in a region between a first region and a second region, and the convex portions may be fitted into two holes of the base plate.

One of two holes of a base plate may be a long hole.

The medical observation system may further include a transmission unit that directly or indirectly transmits an output of the first imaging element, an output of the second imaging element, an output of the third imaging element, and an output of the fourth imaging element to an image generation unit that generates a three-dimensional image on the basis of the output of the first imaging element, the output of the second imaging element, the output of the third imaging element, and the output of the fourth imaging element.

The medical observation system may further include: a first optical member that is disposed on a side of a first surface and transmits the light of the first wavelength band in the first optical path and the second optical path;
a second optical member that transmits the light of the first wavelength band in the first optical path to a side of the first surface that is a spectral surface and reflects the light of the second wavelength band to a side of a second surface, and transmits the light of the first wavelength band in the second optical path to the side of the first surface and reflects the light of the second wavelength band in the second optical path to the side of the second surface; and
a band limiting unit that is disposed on the side of the first surface, transmits the light of the first wavelength band to the side of the first surface, and reflects the light of the second wavelength band to the side of the second surface.

The spectroscopic dispersion of the light in the first optical path and the spectroscopic dispersion of the light in the second optical path may be performed by the same band limiting unit arranged on the spectral surface. A first imaging element that images the light in the first optical path and a third imaging element that images the light in the second optical path may be arranged on a third surface of the first optical member, and
a second imaging element that images the light in the first optical path and a fourth imaging element that images the light in the second optical path may be arranged on the second surface of the second optical member.

### BRIEF DESCRIPTION OF DRAWINGS

FIG. 1 is a diagram illustrating a configuration of a medical observation system according to the present embodiment.

FIG. 2 is a block diagram illustrating a configuration of a camera head and a control device.

FIG. 3 is a top view of a camera head.

FIGS. 4A to 4C are diagrams illustrating a configuration example of a spectroscopic element.

FIG. 5 is a diagram illustrating an optical path in a left side view of the camera head.

FIG. 6 is a top view of a spectroscopic element according to a comparative example.

FIGS. 7A to 7D are diagrams illustrating a process of manufacturing the spectroscopic element according to the present embodiment.

FIGS. 8A to 8D are diagrams illustrating a process of manufacturing a spectroscopic element according to a comparative example.

FIGS. 9A and 9C to 9E are diagrams illustrating an example of bonding a spectroscopic element and a base plate according to a second embodiment.

FIGS. 10A and 10C to 10E are diagrams illustrating an example of bonding a spectroscopic element and a base plate according to a third embodiment.

FIGS. 11A and 11C to 11E are diagrams illustrating an example of bonding a spectroscopic element and a base plate according to a fourth embodiment.

### DESCRIPTION OF EMBODIMENTS

Hereinafter, embodiments of the present disclosure will be described with reference to the drawings. Hereinafter, modes for carrying out the present disclosure (hereinafter referred to as embodiments) will be described with reference to the drawings. Note that the present disclosure is not limited by the embodiments described below. Furthermore, in the description of the drawings, the same portions are denoted by the same reference numerals.

### (Schematic Configuration of Medical Observation System)

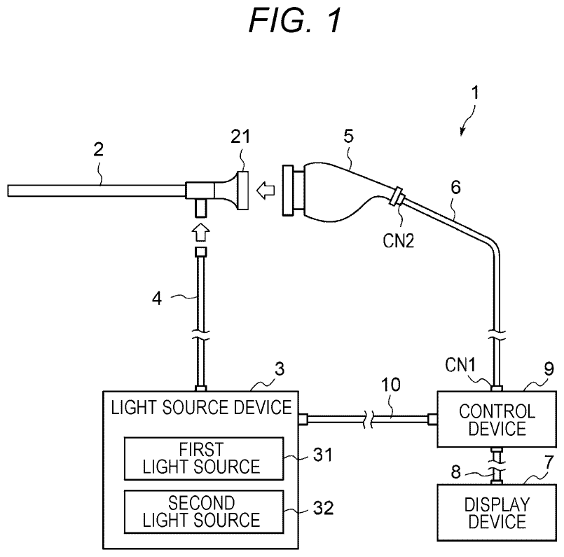

FIG. 1 is a diagram illustrating a configuration of a medical observation system 1 according to the present embodiment.

The medical observation system 1 is a system that is used in a medical field and images (observes) the inside of a living body (observation target) that is a subject. As illustrated in FIG. 1, the medical observation system 1 includes an insertion unit 2, a light source device 3, a light guide 4, a camera head 5, a first transmission cable 6, a display device 7, a second transmission cable 8, a control device 9, and a third transmission cable 10. The medical observation system 1 according to the present embodiment has an observation mode for observing light in a first wavelength band and an observation mode for imaging light in a second wavelength band different from the first wavelength band. In addition, the medical observation system 1 may be configured to have an observation mode for imaging light in a third wavelength band different from the first wavelength band and the second wavelength band. Further, the medical observation system 1 may include an observation mode for observing the light of the first wavelength band and the light of the second wavelength band, an observation mode for observing the light of the first wavelength band and the light of the third wavelength band, and an observation mode for observing the light of the first wavelength band, the light of the second wavelength band, and the light of the third wavelength band. Here, “different wavelength bands” means that the entire two wavelength bands do not completely match.

In the present embodiment, a normal light observation mode for observing white light (normal light) in a first wavelength band, a first fluorescence observation mode for observing white light and first fluorescence in a second wavelength band at least a part of which is outside the wavelength band of white light, and a second fluorescence observation mode for observing white light and second fluorescence in a third wavelength band at least a part of which is within the wavelength band of white light will be described in particular detail. However, the present disclosure is not limited thereto, and the medical observation system 1 can perform observation in a combination of an arbitrary wavelength band and an arbitrary wavelength band. In addition, the control device 9 may change a wavelength band observed by the medical observation system 1 or a combination of wavelength bands on the basis of selection or a program by the user.

The insertion unit 2 is configured by, for example, a binocular relay system (rigid endoscope). In this binocular relay type scope, two optical paths are arranged in parallel in the scope. Furthermore, an optical system is disposed on each of the two optical paths. Then, the scope of the binocular relay system takes in and emits the observation light for the left eye and the observation light for the right eye having parallax with each other by the two optical systems. Note that the medical observation system 1 according to the present embodiment will be described as an example of a binocular relay system, but is not limited thereto. For example, the insertion unit 2 may include a monocular pupil division type scope.

In the monocular pupil division type scope, one optical path is provided in the scope. Furthermore, an optical system is disposed in one optical path. Furthermore, a pupil division portion that divides a light flux in the pupil into two regions is provided at a pupil position of the optical system. Then, in the scope of the monocular pupil division system, the observation light is captured by the optical system, and the observation light is separated into the left and right eye observation light having parallax with each other and emitted by the pupil division unit.

The light source device 3 is connected to one end of the light guide 4, and supplies light to irradiate the inside of the living body to the one end of the light guide 4 under the control of the control device 9. As illustrated in FIG. 1, the light source device 3 includes a first light source 31 and a second light source 32. In the present embodiment, the first light source 31 includes an element that emits white light (first emission light). As the light emitting element, for example, a light emitting diode (LED) or a laser diode (LD) which is a semiconductor element can be used. Return light of the first emission light from the observation target is light in the first wavelength band. In the present embodiment, the light in the first wavelength band is white light.

In the first fluorescence observation mode, the second light source 32 emits (emits) second emission light (first excitation light) having a wavelength band different from the wavelength band of the first emission light. Alternatively, the second light source 32 emits (emits) third emission light (second excitation light) having a wavelength band different from the wavelength band of the second emission light in the second fluorescence observation mode. In the present embodiment, the second light source 32 includes an element that emits near-infrared excitation light as the second emission light and an element that emits third emission light different from the wavelength band of the second emission light. As the light emitting element, for example, an LED or an LD which is a semiconductor element can be used. The return light of the second emission light from the observation target becomes the first fluorescence (light of the second wavelength band) having the second wavelength band different from the first wavelength band. In addition, the return light of the third emission light from the observation target becomes the second fluorescence (light of the third wavelength band) having the third wavelength band different from the first wavelength band and the second wavelength band. At least a part of the third wavelength band overlaps with a part of the first wavelength band. That is, the second fluorescence is visible fluorescence in which at least a part of the wavelength band is visible light.

The near-infrared excitation light emitted by the second light source 32 is excitation light that excites a fluorescent substance such as indocyanine green. Furthermore, when excited with near-infrared excitation light, a fluorescent substance such as indocyanine green emits fluorescence having a central wavelength on a longer wavelength side than a central wavelength of a wavelength band of the near-infrared excitation light. Note that the wavelength band of the near-infrared excitation light and the wavelength band of the fluorescence may be set so as to partially overlap each other, or may be set so as not to overlap each other at all.

Examples of the fluorescent substance contained in the observation target excited by the first excitation light or the second excitation light include a drug or a fluorescent dye applied to the observation target, or a fluorescent substance derived from the observation target configuring the observation target itself.

Examples of the above-described drug given to the observation target include “5-ALA (PP-IX)”, “ADS780WS”, “ADS830WS”, “aggregation-induced emission dots allophycocyanin (APC)”, “boron-dipyrromethane (BODIPY)”, “CLR 1502”, “Flavins”, “fluorescamine”, “Fluorescein”, “fluoro-gold”, “green fluorescence protein”, “ICG (indocyanine green)”, “IRDye 78”, “IR-PEG nanoparticles”, “Isothiocyanate”, “rose Bengal”, “SGM-101”, and “trypan blue”.

In addition, the above-described fluorescent dyes to be imparted to the observation target include “coumarine”, “Cy3”, “DyLight547”, “GE3126”, “metal nanoclusters”, “oxacarbocyanine”, “Rhodamine”, “Riboflavin”, “Fluorescein”, “AlexaFluor 488”, “AlexaFluor660”, “AlexaFluor680”, “AlexaFluor700”, “Cy5”, “Cy5.5”, “Dy677”, “Dy682”, “Dy752”, “DyLight647”, “HiLyte Fluor 647”, “HiLyte Fluor 680”, “IRDye 700DX”, “methylene blue”, “Porphyrins”, “Porphysomes”, “VivoTag-680”, “VivoTag-S680”, “AlexaFluor750”, “AlexaFluor790”, “carbocyanine”, “conjugated copolymers”, “CW800-CA”, “Cy7”, “Cy7.5”, “cyanine dyes”, “Dy780”, “HiLyte Fluor 750”, “Indocarbocyanine”, “IR-786”, “IRDye 800CW”, “IRDye 800RS”, “IRDye 800BK”, “Nervelight”, “OTL-38”, “Polymethine”, “VivoTag-S750”, “ASP5354”, and “Xanthene”. Furthermore, examples of the fluorescent substance derived from the observation target configuring the observation target itself include “collagen”, “elastin”, and “NADH”.

Then, in the light source device 3 according to the present embodiment, the first light source 31 is driven in the normal observation mode under the control of the control device 9. That is, in the normal observation mode, the light source device 3 emits normal light (white light). On the other hand, in the light source device 3, under the control of the control device 9, in the first fluorescence observation mode, the first light source 31 may be driven to emit the first emission light in a first period of the alternately repeated first and second periods, and the second light source 32 may be driven to emit the second emission light in the second period. That is, in the first fluorescence observation mode, the light source device 3 may emit normal light (white light) in the first period and emit near-infrared excitation light in the second period. Furthermore, in the first fluorescence observation mode, the emission of the first emission light by the first light source 31 and the emission of the second emission light by the second light source 32 may be performed simultaneously. Furthermore, in the second fluorescence observation mode, among the alternately repeated first and second periods, the first light source 31 is driven to emit the first emission light in the first period, and the second light source 32 is driven to emit the third emission light in the second period. Note that in the present embodiment, the light source device 3 is configured separately from the control device 9, but the present disclosure is not limited thereto, and a configuration provided inside the control device 9 may be adopted.

One end of the light guide 4 is detachably connected to the light source device 3, and the other end is detachably connected to the insertion unit 2. Then, the light guide 4 transmits light (normal light or near-infrared excitation light) supplied from the light source device 3 from one end to the other end, and supplies the light to the insertion unit 2. In a case where the normal light (white light) is emitted into the living body, the normal light in the first wavelength band reflected in the living body is condensed in the insertion unit 2.

In addition, in a case where the near-infrared excitation light is emitted into the living body, the near-infrared excitation light reflected in the living body and a fluorescent substance such as indocyanine green that accumulates at a lesion in the living body are excited, and fluorescence emitted from the fluorescent substance is condensed in the insertion unit 2. That is, the first excitation light and the first fluorescence in the second wavelength band different from the first wavelength band are condensed in the insertion unit 2.

Note that, hereinafter, for convenience of description, normal light that is left and right eye observation light condensed in the insertion unit 2 and emitted from the insertion unit 2 is referred to as first left and right eye subject images. In addition, near infrared excitation light and fluorescence, which are left and right-eye observation light collected in the insertion unit 2 and emitted from the insertion unit 2, are referred to as second left and right-eye subject images, respectively.

The camera head 5 corresponds to an imaging device according to the present disclosure. The camera head 5 is detachably connected to the proximal end (eyepiece unit 21 (FIG. 1)) of the insertion unit 2. Then, under the control of the control device 9, the camera head 5 captures the first left and right-eye subject images (normal light) and the second left and right-eye subject images (near-infrared excitation light and fluorescence) emitted from the insertion unit 2, and outputs an image signal by imaging. Note that a detailed configuration of the camera head 5 will be described later.

One end of the first transmission cable 6 is detachably connected to the control device 9 through a connector CN1 (FIG. 1), and the other end is detachably connected to the camera head 5 through a connector CN2 (FIG. 1). Then, the first transmission cable 6 transmits an image signal and the like output from the camera head 5 to the control device 9, and transmits a control signal, a synchronization signal, a clock, power, and the like output from the control device 9 to the camera head 5. Note that in the transmission of the image signal and the like from the camera head 5 to the control device 9 through the first transmission cable 6, the image signal and the like may be transmitted as an optical signal or may be transmitted as an electric signal. The same applies to transmission of a control signal, a synchronization signal, and a clock from the control device 9 to the camera head 5 through the first transmission cable 6.

The display device 7 displays an image based on a video signal from the control device 9. One end of the second transmission cable 8 is detachably connected to the display device 7, and the other end is detachably connected to the control device 9. Then, the second transmission cable 8 transmits the video signal processed by the control device 9 to the display device 7.

The control device 9 corresponds to a medical image processing apparatus according to the present disclosure. The control device 9 includes a central processing unit (CPU), a field-programmable gate array (FPGA), and the like, and integrally controls operations of the light source device 3, the camera head 5, and the display device 7. Note that a detailed configuration of the control device 9 will be described later.

One end of the third transmission cable 10 is detachably connected to the light source device 3, and the other end is detachably connected to the control device 9. Then, the third transmission cable 10 transmits the control signal from the control device 9 to the light source device 3.

### (Configuration of Camera Head 5)

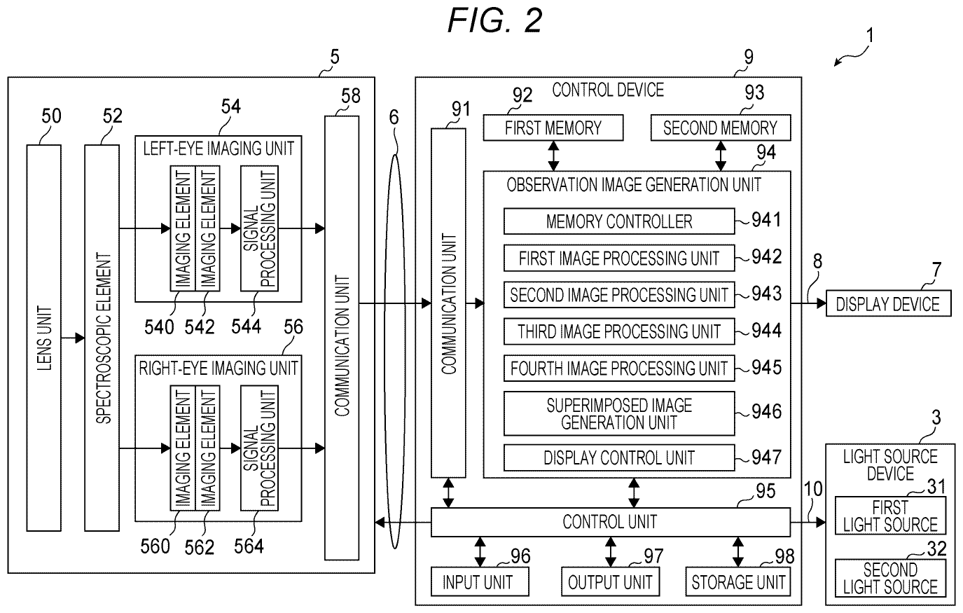

Configurations of the camera head 5 and the control device 9 will be described with reference to FIG. 2. FIG. 2 is a block diagram illustrating configurations of the camera head 5 and the control device 9. Note that details of a configuration example of the optical system of the camera head 5 will be described later.

In FIG. 2, for convenience of description, the connectors CN1 and CN2 between the control device 9 and the camera head 5 and the first transmission cable 6, the connectors between the control device 9 and the display device 7 and the second transmission cable 8, and the connectors between the control device 9 and the light source device 3 and the third transmission cable 10 are omitted.

As illustrated in FIG. 2, the camera head 5 includes a lens unit 50, a spectroscopic element 52, left and right eye imaging units 54 and 56, and a communication unit 58. The left-eye imaging unit 54 captures the first left-eye subject image (normal light) and the second left-eye subject image (first fluorescence) emitted from the insertion unit 2 under the control of the control device 9. Furthermore, the left-eye imaging unit 54 can be configured to capture a third left-eye subject image (second fluorescence). The left-eye imaging unit 54 includes imaging elements 540 and 542 and a signal processing unit 544. The imaging element 540 captures a first left-eye subject image (normal light) and a third left-eye subject image (second fluorescence). The imaging element 542 captures the second left-eye subject image (first fluorescence).

The right-eye imaging unit 56 captures the first right-eye subject image (normal light) and the second right-eye subject image (first fluorescence) emitted from the insertion unit 2 under the control of the control device 9. Furthermore, the right-eye imaging unit 56 can be configured to capture a third right-eye subject image (second fluorescence). As illustrated in FIG. 2, the right-eye imaging unit 56 includes imaging elements 560 and 562 and a signal processing unit 564. The imaging element 560 captures a first right-eye subject image (normal light) and a third right-eye subject image (second fluorescence). The imaging element 562 captures a second right-eye subject image (first fluorescence). An excitation light cut filter (band-pass filter) may be provided at a position on the insertion unit 2 side with respect to the imaging element 542 in the insertion unit 2 or the camera head 5. In this case, the second left-eye subject image, the third left-eye subject image, the second right-eye subject image, and the third right-eye subject image captured by the left-eye imaging unit 54 and the right-eye imaging unit 55 are substantially or completely only fluorescent. However, the present disclosure is not limited thereto, and the left-eye imaging unit 54 and the right-eye imaging unit 55 may be configured to image a part or all of the first excitation light and the second excitation light together with the first fluorescence and the second fluorescence by providing no excitation light cut filter or adjusting a ratio of the excitation light cut by the excitation light cut filter.

The lens unit 50 forms the first and second left-eye subject images and the first and second right-eye subject images emitted from the insertion unit 2 on the imaging elements 540 and 542 and the imaging elements 560 and 562 through the spectroscopic element 52. The spectroscopic element 52 spectrally disperses light of one optical path for the left eye into light of a first wavelength band and light of a second wavelength band different from the first wavelength band, and spectrally disperses light of a second optical path different from the first optical path for the right eye into light of the first wavelength band and light of the second wavelength band.

Furthermore, the lens unit 50 can be configured to form the third left-eye subject image and the third right-eye subject image emitted from the insertion unit 2 on the imaging element 540 and the imaging element 560 through the spectroscopic element 52.

The imaging elements 540 and 542 and the imaging elements 560 and 562 include a charge coupled device (CCD), a complementary metal oxide semiconductor (CMOS), or the like that receives light and converts the light into an electrical signal (analog signal). The imaging element 540 images light in a first wavelength band for the left eye. Furthermore, the imaging element 540 can be configured to image light in the third wavelength band for the left eye. The imaging element 542 images the light of the second wavelength band for the left eye through the band-pass filter. Here, band-pass filters are provided on imaging surfaces (light receiving surfaces) of the imaging elements 542 and 562. The band-pass filter transmits light in a wavelength band corresponding to fluorescence among near-infrared excitation light and fluorescence.

Similarly, the imaging element 560 images light in the first wavelength band for the right eye. Furthermore, the imaging element 560 can be configured to image light in the third wavelength band for the right eye. The imaging element 562 images the light of the second wavelength band for the right eye through the band-pass filter.

The imaging element 540 captures the first left-eye subject image (normal light) at a predetermined frame rate in the normal observation mode under the control of the control device 9. Furthermore, under the control of the control device 9, the imaging elements 540 and 542 perform imaging every first and second periods that are alternately repeated in synchronization with the light emission timing of the alternate light emission of the first emission light and the second emission light of the light source device 3 in the first fluorescence observation mode. Furthermore, in the first fluorescence observation mode, the imaging elements 540 and 542 may simultaneously image the light of the first wavelength band and the light of the second wavelength band in synchronization with the simultaneous emission of the first emission light and the second emission light by the light source device 3. Furthermore, the imaging element 540 may perform imaging in synchronization with the light emission timing of the alternate light emission of the first emission light and the third emission light of the light source device 3 in the second fluorescence observation mode under the control of the control device 9.

Similarly, the imaging element 560 captures the first right-eye subject image (normal light) at a predetermined frame rate in the normal observation mode under the control of the control device 9. Furthermore, under the control of the control device 9, the imaging elements 560 and 562 perform imaging every first and second periods that are alternately repeated in synchronization with the light emission timing of the alternate light emission of the first emission light and the second emission light of the light source device 3 in the first fluorescence observation mode. Furthermore, in the first fluorescence observation mode, the imaging elements 560 and 562 may simultaneously image the light of the first wavelength band and the light of the second wavelength band in synchronization with the simultaneous emission of the first emission light and the second emission light by the light source device 3. Furthermore, the imaging element 560 may perform imaging in synchronization with the light emission timing of the alternate light emission of the first emission light and the third emission light of the light source device 3 in the second fluorescence observation mode under the control of the control device 9.

The communication unit 58 functions as a transmitter that transmits the left-eye captured image in raster units sequentially output from the left-eye imaging unit 54 and the right-eye captured image in raster units sequentially output from the right-eye imaging unit 56 to the control device 9 through the first transmission cable 6. That is, the communication unit 58 directly or indirectly transmits the outputs of the imaging elements 540 and 542 and the output imaging elements 560 and 562 to the observation image generation unit 94 of the control device 9. Note that the communication unit 58 according to the present embodiment corresponds to a transmission unit.

Note that, in the present embodiment, the configuration in which the camera head 5 connected to the proximal end of the insertion unit 2 of the rigid endoscope includes the lens unit 50, the spectroscopic element 52, and the left and right eye imaging units 54 and 56 has been described. However, the present disclosure is not limited thereto, and the lens unit 50, the spectroscopic element 52, and the left and right eye imaging units 54 and 56 may be provided at the distal end of the insertion unit. In addition, the present disclosure may be applied to a flexible endoscope.

### (Configuration of Control Device)

Next, a configuration of the control device 9 will be described with reference to FIG. 2.

As illustrated in FIG. 2, the control device 9 includes a communication unit 91, first and second memories 92 and 93, an observation image generation unit 94, a control unit 95, an input unit 96, an output unit 97, and a storage unit 98. The communication unit 91 functions as a receiver that receives the left and right eye captured images in raster units sequentially output from the camera head 5 (communication unit 58) through the first transmission cable 6. The communication unit 91 corresponds to a first captured image acquisition unit and a second captured image acquisition unit according to the present disclosure.

The first memory 92 temporarily stores the left and right eye captured images sequentially output from the camera head 5 (communication unit 58). The second memory 93 temporarily stores the image processed by the observation image generation unit 94.

The observation image generation unit 94 processes the left and right eye captured images in raster units sequentially output from the camera head 5 (communication unit 58) and received by the communication unit 91 under the control of the control unit 95. As illustrated in FIG. 2, the observation image generation unit 94 includes a memory controller 941, first to fourth image processing units 942 to 945, a superimposed image generation unit 946, and a display control unit 947.

The memory controller 941 controls writing of an image to the first memory 92 and reading of an image from the first memory 92 under the control of the control unit 95. Note that details of the function of the memory controller 941 will be described in “operation of control device” described later.

The first to fourth image processing units 942 to 945 execute image processing in parallel on each input image under the control of the control unit 95. Note that the configurations of the first to fourth image processing units 942 to 945 are the same. The first image processing unit 942 processes the image signal of the imaging element 540, the second image processing unit 943 processes the image signal of the imaging element 542, the third image processing unit 944 processes the image signal of the imaging element 560, and the fourth image processing unit 945 processes the image signal of the imaging element 562.

The superimposed image generation unit 946 operates in the first fluorescence observation mode and the second fluorescence observation mode under the control of the control unit 95. Then, the superimposed image generation unit 946 generates fluorescence superimposed images for the left and right eyes on the basis of the images after the image processing is executed by the first to fourth image processing units 942 to 945.

The display control unit 947 generates a three-dimensional image of white light or second fluorescence for display from the image after the image processing is executed by the first image processing unit 942 and the third image processing unit 944. In addition, the display control unit 947 generates a three-dimensional image of the first fluorescence for display from the image after the image processing is executed by the second image processing unit 943 and the fourth image processing unit 945. Furthermore, the display control unit 947 generates a three-dimensional image for display from the image after the image processing is executed by the first to fourth image processing units 942 to 945 and the left and right eye fluorescence superimposed images generated by the superimposed image generation unit 946. Then, the display control unit 947 outputs a video signal for displaying a three-dimensional image to the display device 7 through the second transmission cable 8. In addition, the display control unit 947 may generate a three-dimensional image in which the information of the white light and the information of the first fluorescence and/or the second fluorescence are combined from the image after the image processing is executed by the first image processing unit 942, the second image processing unit 943, the third image processing unit 944, and the fourth image processing unit 945 without passing through the process of generating a three-dimensional image of the white light and the process of generating a three-dimensional image of the first fluorescence and/or the second fluorescence.

The control unit 95 is configured using, for example, a CPU, an FPGA, or the like, and outputs a control signal through the first to third transmission cables 6, 8, and 10, thereby controlling the operations of the light source device 3, the camera head 5, and the display device 7, and controlling the entire operation of the control device 9.

The input unit 96 is configured using an operation device such as a mouse, a keyboard, and a touch panel, and receives a user operation by a user such as a doctor. Then, the input unit 96 outputs an operation signal corresponding to the user operation to the control unit 95.

The output unit 97 is configured using a speaker, a touch panel, or the like, and outputs various types of information. The storage unit 98 stores a program executed by the control unit 95, information necessary for processing of the control unit 95, and the like.

### (Configuration of Optical System of Camera Head 5)

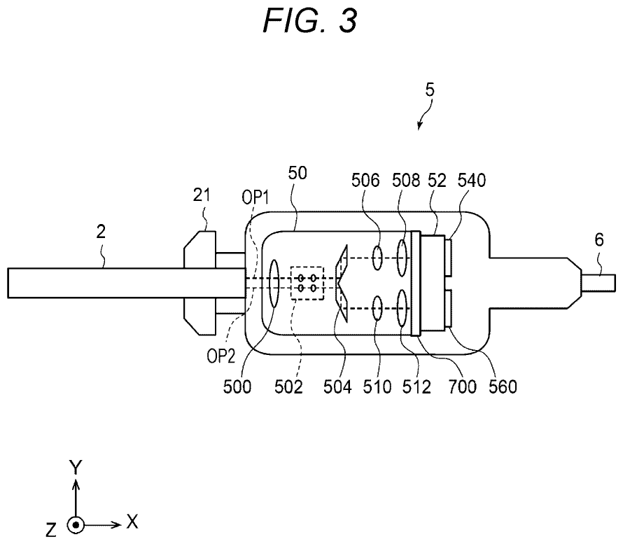

FIG. 3 is a top view of the camera head 5. Inside the insertion unit 2, first and second optical paths OP1 and OP2 extending along the central axis of the insertion unit 2 and arranged in parallel to each other so as to be symmetric about the central axis are set. The first and second optical paths OP1 and OP2 are disposed at regular intervals in the radial direction inside the insertion unit 2. Therefore, the insertion unit 2 takes in and emits the first and second observation light having parallax with each other.

The lens unit 50 includes an objective optical system 500, a relay optical system 502, a prism 504, first imaging optical systems 506 and 508, and second imaging optical systems 510 and 512. The objective optical system 500 captures the first and second observation light and emits the first and second observation light to the relay optical system 502. The relay optical system 502 has a pair of a first optical path OP1 side and a second optical path OP2 side.

The relay optical system 502 on the first optical path OP1 side captures the first observation light and emits the first observation light to the first optical path OP1 side of the prism 504. Similarly, the relay optical system 502 on the second optical path OP2 side takes in the second observation light and emits the second observation light to the second optical path OP2 side of the prism 504.

The first imaging optical systems 506 and 508 form images of the first observation light on the first optical path OP1 side on the imaging elements 540 and 542 through the spectroscopic element 52. Similarly, the second imaging optical systems 510 and 512 form images of the second observation light on the second optical path OP2 side on the imaging elements 560 and 562 through the spectroscopic element 52.

### (Configuration of Spectroscopic Element 52)

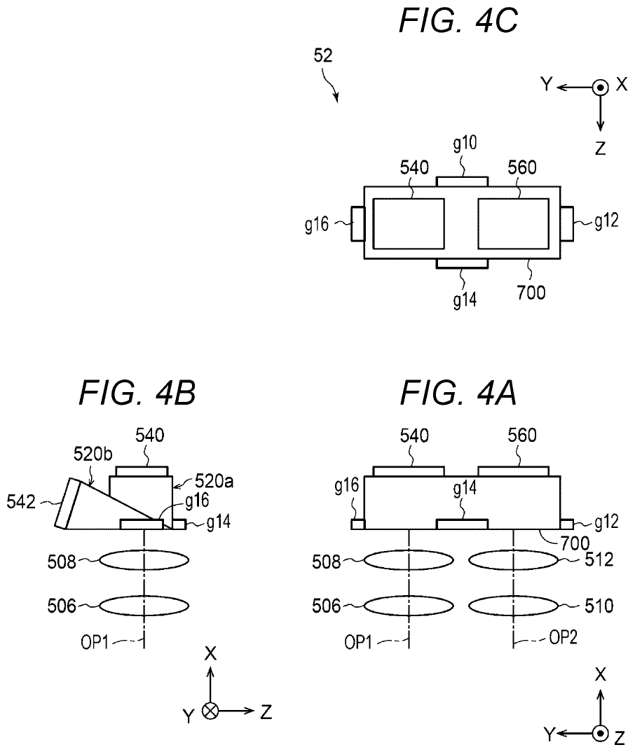

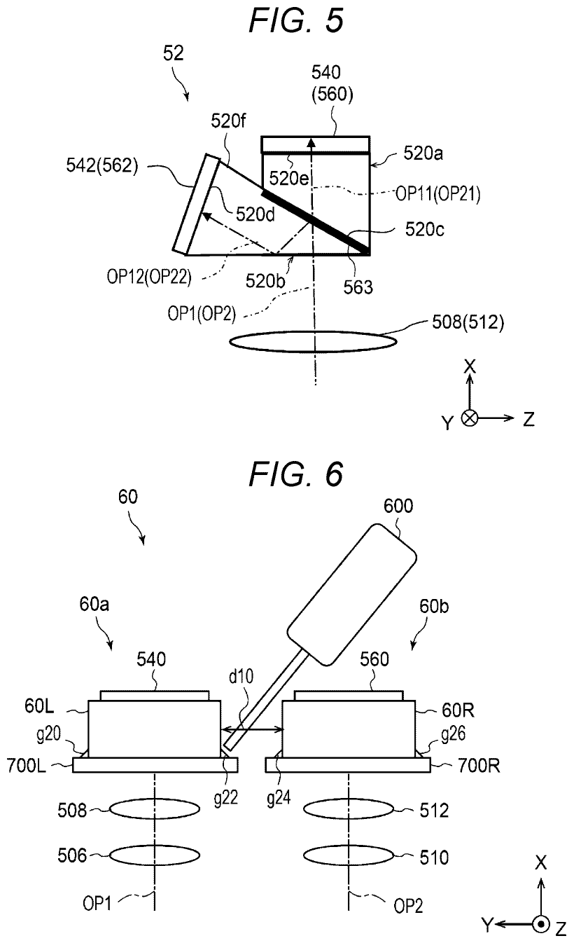

A configuration example of the spectroscopic element 52 of the camera head 5 will be described in detail with reference to FIGS. 4A to 4C and 5. FIGS. 4A to 4C are diagrams illustrating a configuration example of the spectroscopic element 52. FIG. 4A is a top view of the camera head 5. FIG. 4B is a left side view of the camera head 5. FIG. 4C is a rear view of the camera head 5. FIG. 5 is a diagram illustrating an optical path in a left side view of the camera head 5.

As illustrated in FIGS. 4A to 4C and 5, the spectroscopic element 52 includes a first optical member 520a including a transparent material, a second optical member 520b including a transparent material, and a dichroic mirror 563. The transparent first optical member 520a and second optical member 520b configure a prism. The dichroic mirror 563 is provided between a first surface 520c of first optical member 520a and a fourth surface 520f of the second optical member 520b. The dichroic mirror 563 may also serve as a bonding layer for bonding the first optical member 520a and the second optical member 520b, or a bonding layer may be provided as a layer different from the dichroic mirror 563. In a case where the bonding layer is provided as a layer different from the dichroic mirror 563, the dichroic mirror 563 may be provided on the first surface 520c of the first optical member 520a or may be provided on the fourth surface 520f of the second optical member 520b. Note that the dichroic mirror 563 according to the present embodiment corresponds to a band limiting unit.

In addition, the imaging elements 540 and 560 are arranged on the second surface 520d that is the same surface of the first optical member 520a, and the imaging elements 542 and 562 are arranged on the third surface 520e that is the same surface of the second optical member 520b. In addition, the spectroscopic element 52 is fixed to a base plate 700 including a transparent material by adhesive portions g10 to g16.

As illustrated in FIG. 5, out of the first observation light on the first optical path OP1 side and the second observation light on the second optical path OP2 side, which have passed through the lens 508 (512), the light of the second wavelength band (fluorescence) is reflected by the dichroic mirror 563 and guided to the imaging element 542 (562). On the other hand, the first wavelength band (visible light) of the light transmitted through the lens 508 (512) is transmitted through the dichroic mirror 563 and guided to the imaging elements 540 and 560. That is, the spectroscopic dispersion of the first optical path OP1 and the spectroscopic dispersion of the second optical path OP2 are executed by the integrally formed spectroscopic element 52.

As described above, the spectroscopic element 52 spectrally disperses the light of the first optical path OP1 into an optical path OP11 of the first wavelength band (visible light) and an optical path OP12 of the second wavelength band (fluorescence), and spectrally disperses the light of the second optical path OP2 into an optical path OP21 of the first wavelength band (visible light) and an optical path OP22 of the second wavelength band (first fluorescence). Moreover, the surface (spectral surface) of the dichroic mirror 563 that spectrally disperses the light of the first optical path OP1 and the surface (spectral surface) of the dichroic mirror 563 that spectrally disperses the light of the second optical path OP2 are both configured on the same plane of the same spectroscopic element 52.

Moreover, in a case where the light of the third wavelength band (visible fluorescence) is included in the light of the first optical path OP1 and the second optical path OP2, the spectroscopic element 52 spectrally disperses the light of the first optical path OP1 and the second optical path OP2 so that the light of the third wavelength band advances to the optical path OP11 and the optical path OP21.

FIG. 6 is a top view of the spectroscopic element 60 according to a comparative example. A spectroscopic element 600 according to the comparative example generally includes a spectroscopic element 60a for the first optical path OP1 and a spectroscopic element 60b for the second optical path OP2. The spectroscopic element 60a and the spectroscopic element 60b are separated. In addition, four sides of the spectroscopic element 60a are fixed to a base plate 700a by the bonding portion application jig 600 at bonding portions g20 to g22. Similarly, four sides of the spectroscopic element 60b are fixed to a base plate 700b by the bonding portion application jig 600 at bonding portions g24 to g26.

At this time, in order to apply the bonding portions g22 and g24 between the spectroscopic element 60a and the spectroscopic element 60b, a work space of the bonding portion application jig 600 is required. Therefore, an interval d10 for securing the work space is required between the spectroscopic element 60a and the spectroscopic element 60b. That is, a length of the spectroscopic element 60 in a y direction becomes longer by the interval d10. On the other hand, since the spectroscopic element 52 according to the present embodiment is integrally formed, the adhesion portions g22 and g24 between the spectroscopic element 60a and the spectroscopic element 60b are unnecessary. In addition, the interval d10 for applying the bonding portions g22 and g24 becomes unnecessary. As a result, the spectroscopic element 52 according to the present embodiment can be further downsized.

Furthermore, in a case where the spectroscopic element 60a and the spectroscopic element 60b are made independent of each other, arrangement accuracy for making arrangement surfaces of the imaging element 540 and the imaging element 560 the same on the optical path is required. Similarly, there is a demand for arrangement accuracy for making arrangement surfaces of the imaging element 542 (see FIG. 5) and the imaging element 562 (see FIG. 5) the same on the optical path. Similarly, the arrangement accuracy is also required to equalize the spectral surface that spectrally disperses the light of the first optical path OP1 and the spectral surface that spectrally disperses the light of the second optical path OP2 on the optical path. On the other hand, since the spectroscopic element 52 according to the present embodiment is integrally formed, it is not necessary to perform such arrangement, and the image quality at the time of observation can be further improved.

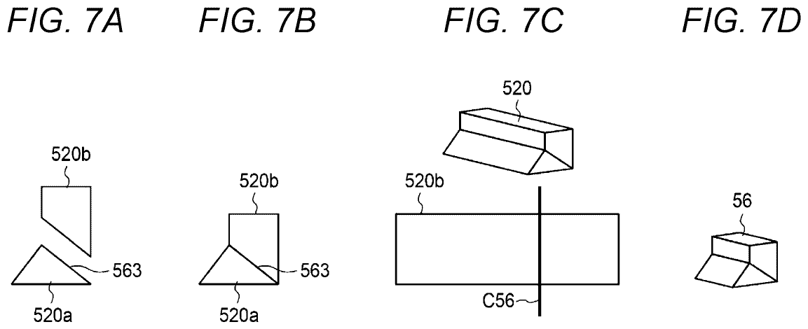

FIGS. 7A to 7D are diagrams illustrating a process of manufacturing the spectroscopic element 52 according to the present embodiment. FIG. 7A illustrates a process of manufacturing the dichroic mirror 563. As illustrated in FIG. 7A, the dichroic mirror 563 is formed on a bonding surface of the first optical member 520a.

FIG. 7B illustrates a process of bonding the first optical member 520a, the second optical member 520b, and the dichroic mirror 563. As illustrated in FIG. 7B, the first optical member 520a, the second optical member 520b, and the dichroic mirror 563 are integrated to form a structure 520.

FIG. 7C illustrates a process of separating the spectroscopic element 52. As illustrated in FIG. 7C, the structure 520 is cut along a cut line C56, and the spectroscopic element 52 illustrated in FIG. 7D is manufactured.

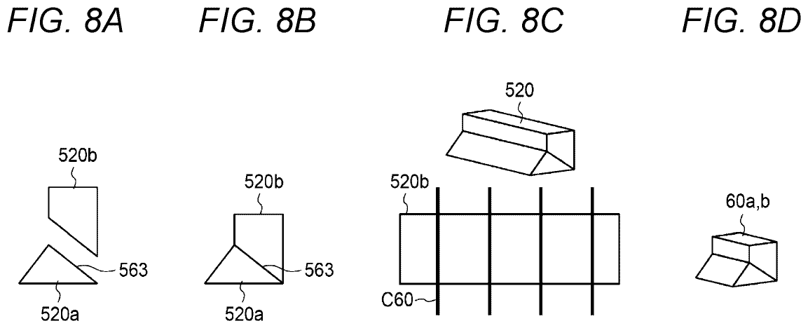

FIGS. 8A to 8D are diagrams illustrating a process of manufacturing the spectroscopic element 60 according to a comparative example. The manufacturing process of FIGS. 8A and 8B corresponds to that of FIGS. 7A and 7B.

FIG. 8C illustrates a process of separating the spectroscopic element 60. As illustrated in FIG. 8C, the structure 520 is cut along a cut line C60, and the spectroscopic elements 60a and 60b illustrated in FIG. 8D are manufactured. As illustrated in FIGS. 8A to 8D, in order to eliminate the optical path difference between the left and right eyes, it is necessary to select the spectroscopic elements 60a and 60b having the same optical characteristics. On the other hand, since the spectroscopic element 52 according to the present embodiment is integrally formed, it is not necessary to perform such selection, and the image quality at the time of observation can be further improved.

As described above, according to the present embodiment, since the prisms 52a and 52b of the spectroscopic element 52 are integrated by the left eye and the right eye, the work space of the adhesive portion application jig between the prisms is unnecessary, and the imaging device can be downsized. Furthermore, adjustment of an optical path difference between the left and right eyes becomes unnecessary, and the image quality at the time of observation can be further improved.

### Second Embodiment

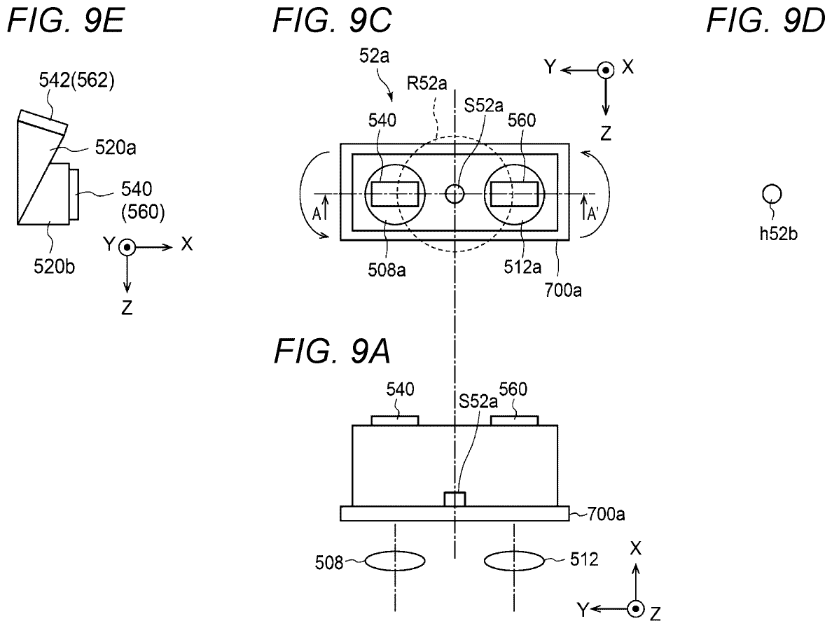

A medical observation system 1 according to a second embodiment is different from the medical observation system 1 according to the first embodiment in that a spectroscopic element 52 and a base plate 700 are bonded by a convex portion of a base plate 700. Hereinafter, differences from the medical observation system 1 according to the first embodiment will be described. FIGS. 9A and 9C to 9E are diagrams illustrating an example of bonding a spectroscopic element 52a and a base plate 700a according to the second embodiment. FIG. 9C is a rear view of the spectroscopic element 52a, and FIG. 9A is an AA′ cross section of FIG. 9C. FIG. 9D is a diagram illustrating a hole shape of the spectroscopic element 52a. FIG. 9E is a left side view of the spectroscopic element 52a.

As illustrated in FIGS. 9A and 9C to 9E, the spectroscopic element 52a includes a first region 508a corresponding to a lens 508 through which light imaged by at least one of a first imaging element 540 or the second imaging element 542 passes, and a second region 512a corresponding to a lens 512 through which light imaged by at least one of a third imaging element 560 or a fourth imaging element 562 passes. As illustrated in FIG. 9D, the spectroscopic element 52a is provided with a hole h52b in a region between the first region and the second region, and a convex portion S52a of a base plate 700a for fixing the spectroscopic element 52a is fitted into the hole h52b.

By restricting the movement of the spectroscopic element 52a up, down, left, and right in this manner, it is possible to reduce a problem that the spectroscopic element 52a moves due to temperature or the like.

Furthermore, a circle R52a centered on the center of the hole h52b passes through the optical axis of a lens 508 and the optical axis of a lens 512. That is, even if movement occurs in the spectroscopic element 52a, the optical axis of the lens 508 and the optical axis of the lens 512 are configured to be equidistant from the center of the hole h52b, and deterioration in image quality at the time of observation is suppressed.

### Third Embodiment

A medical observation system 1 according to a third embodiment is different from the medical observation system 1 according to the second embodiment in that a spectroscopic element 52b and a base plate 700b are bonded by two convex portions of a base plate 700. Hereinafter, differences from the medical observation system 1 according to the second embodiment will be described.

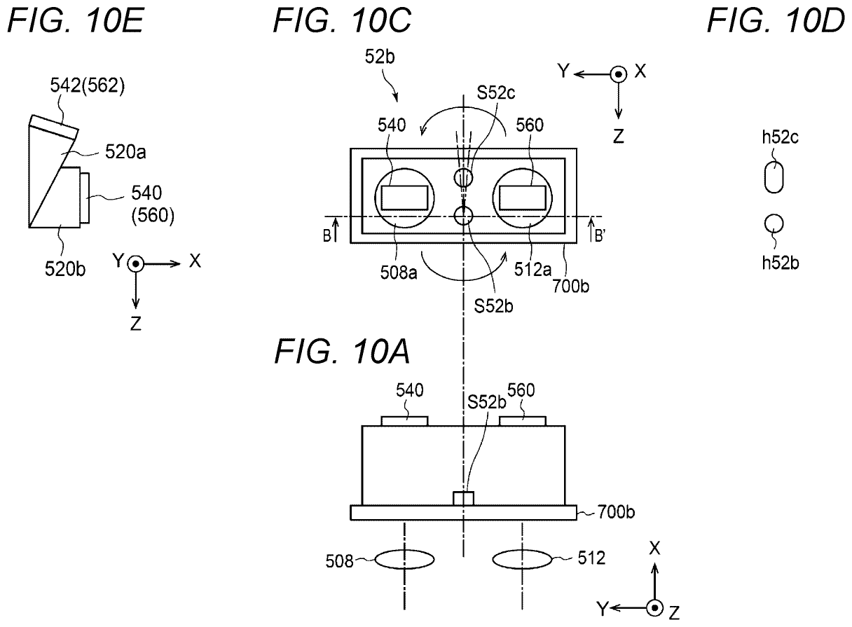

FIGS. 10A and 10C to 10E are diagrams illustrating an example of bonding the spectroscopic element 52b and the base plate 700b according to the third embodiment. FIG. 10C is a rear view of the spectroscopic element 52b, and FIG. 10A is a BB′ cross section of FIG. 10C. FIG. 10D is a diagram illustrating a hole shape of the spectroscopic element 52b. FIG. 9E is a left side view of the spectroscopic element 52b.

As illustrated in FIGS. 10A and 10C to 10E, the spectroscopic element 52b includes a first region 508a corresponding to the lens 508 through which light imaged by at least one of the first imaging element 540 or the second imaging element 542 passes, and a second region 512a corresponding to the lens 512 through which light imaged by at least one of the third imaging element 560 or the fourth imaging element 562 passes. As illustrated in FIG. 10D, the spectroscopic element 52b is provided with holes h52b and h52c in a region between a first region and a second region, and convex portions S52b and S52c of the base plate 700a for fixing the spectroscopic element 52a are fitted into the holes h52b and h52c. One of the holes h52b and h52c is a long hole.

Thus, the spectroscopic element 52b is fixed by the two convex portions S52b and S52c. As a result, the movement of the spectroscopic element 52b in the vertical and horizontal directions is restricted, and a problem that the spectroscopic element 52b moves due to temperature or the like can be reduced. In addition, by making one of the holes h52b and h52c a long hole, the spectroscopic element 52a and the base plate 700a are more easily bonded.

### Fourth Embodiment

A medical observation system 1 according to a fourth embodiment is different from the medical observation system 1 according to the first embodiment in that a spectroscopic element 52c and a base plate 700c are bonded by two convex portions of the spectroscopic element 52c. Hereinafter, differences from the medical observation system 1 according to the first embodiment will be described.

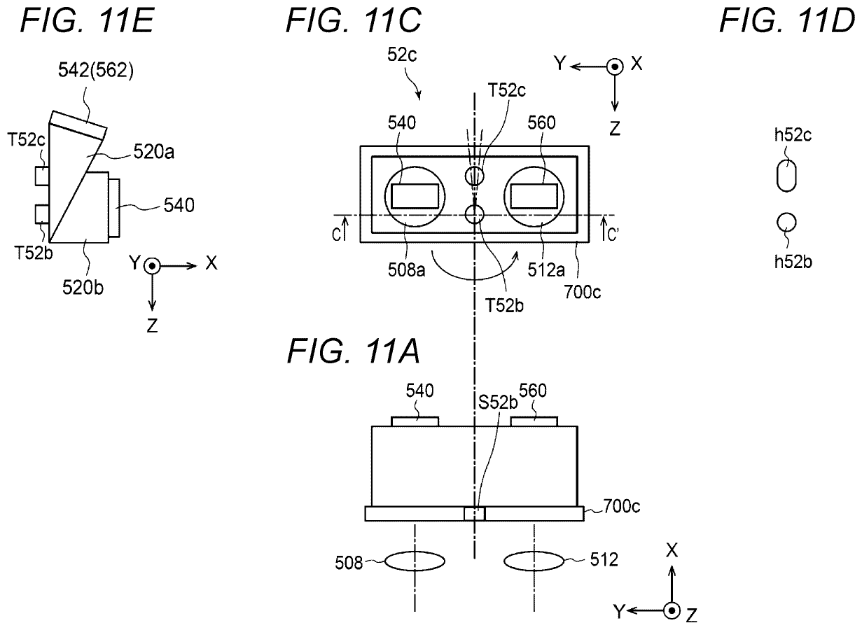

FIGS. 11A and 11C to 11E are diagrams illustrating an example of bonding the spectroscopic element 52c and the base plate 700c according to the fourth embodiment. FIG. 11C is a rear view of the spectroscopic element 52c, and FIG. 11A is a CC′ cross section of FIG. 11C. FIG. 11D is a diagram illustrating a hole shape of the base plate 700c. FIG. 11E is a left side view of the spectroscopic element 52c.

As illustrated in FIGS. 11A and 11C to 11E, the spectroscopic element 52b includes a first region 508a corresponding to the lens 508 through which light imaged by at least one of the first imaging element 540 or the second imaging element 542 passes, and a second region 512a corresponding to the lens 512 through which light imaged by at least one of the third imaging element 560 or the fourth imaging element 562 passes. As illustrated in FIGS. 11C and 11E, in the spectroscopic element 52b, convex portions T52b and T52c are provided in a region between the first region and the second region, and holes h52b and h52c of the base plate 700c for fixing the spectroscopic element 52c are fitted into the convex portions T52b and T52c. One of the holes h52b and h52c is a long hole.

Thus, the spectroscopic element 52c is fixed by the two convex portions T52b and T52c. As a result, the movement of the spectroscopic element 52c in the vertical and horizontal directions is restricted, and a problem that the spectroscopic element 52c moves due to temperature or the like can be reduced. In addition, by forming one of the holes h52b and h52c as a long hole, it is easier to bond the spectroscopic element 52a and the base plate 700a.

The present disclosure can also have the following configurations.

(1)

A medical observation system including:
a spectroscopic element that spectrally disperses light in a first optical path into light of a first wavelength band and light of a second wavelength band different from the first wavelength band, and spectrally disperses light in a second optical path different from the first optical path into light of the first wavelength band and light of the second wavelength band;
a first imaging element that images the light of the first wavelength band spectrally dispersed from the light in the first optical path;
a second imaging element that images the light of the second wavelength band spectrally dispersed from the light in the first optical path;
a third imaging element that images the light of the first wavelength band spectrally dispersed from the light in the second optical path; and
a fourth imaging element that images light of the second wavelength band spectrally dispersed from the light in the second optical path.
   (2)

The medical observation system according to (1), in which the spectroscopic dispersion of the light in the first optical path and the spectroscopic dispersion of the light in the second optical path are performed by the same spectroscopic element.

(3)

The medical observation system according to (2), in which a spectral surface that spectrally disperses the light of the first optical path and a spectral surface that spectrally disperses the light of the second optical path are on the same plane of the same spectroscopic element.

(4)

The medical observation system according to (1), further including an image generation unit that generates a three-dimensional image of an output of the first imaging element, an output of the second imaging element, an output of the third imaging element, and an output of the fourth imaging element on the basis of the output of the first imaging element, the output of the second imaging element, the output of the third imaging element, and the output of the fourth imaging element.

(5)

The medical observation system according to (1), in which the light in the first wavelength band is white light, and the light in the second wavelength band is first fluorescence.

(6)

The medical observation system according to (5), in which when a second fluorescence having a wavelength band different from that of the first fluorescence is imaged, the first imaging element and the third imaging element image white light and the second fluorescence in a time division manner.

(7)

The medical observation system according to (1), in which the spectroscopic element has a first region through which light imaged by at least one of the first imaging element or the second imaging element passes and a second region through which light imaged by at least one of the third imaging element or the fourth imaging element passes, and
in the spectroscopic element, a hole is provided in a region between the first region and the second region, and a convex portion of a base plate for fixing the spectroscopic element is fitted into the hole.
   (8)

The medical observation system according to (7), in which in the spectroscopic element, two of the holes are provided in the region between the first region and the second region, and two of the convex portions of the base plate for fixing the spectroscopic element are fitted into the holes.

(9)

The medical observation system according to (8), in which one of the two holes of the spectroscopic element is a long hole.

(10)

The medical observation system according to (1), in which the spectroscopic element has a first region through which light imaged by at least one of the first imaging element or the second imaging element passes and a second region through which light imaged by at least one of the third imaging element or the fourth imaging element passes, and
in the spectroscopic element, a convex portion is provided in a region between the first region and the second region, and the convex portion is fitted into a hole of a base plate.
   (11)

The medical observation system according to (1), in which in the spectroscopic element, two convex portions are provided in a region between a first region and a second region, and the convex portions are fitted into two holes of the base plate.

(12)

The medical observation system according to (1), in which one of two holes of a base plate is a long hole.

(13)

The medical observation system according to (1), further including a transmission unit that directly or indirectly transmits an output of the first imaging element, an output of the second imaging element, an output of the third imaging element, and an output of the fourth imaging element to an image generation unit that generates a three-dimensional image on the basis of the output of the first imaging element, the output of the second imaging element, the output of the third imaging element, and the output of the fourth imaging element.

(14)

The medical observation system according to (1), further including:
a first optical member that is disposed on a side of a first surface and transmits the light of the first wavelength band in the first optical path and the second optical path;
a second optical member that transmits the light of the first wavelength band in the first optical path to a side of the first surface that is a spectral surface and reflects the light of the second wavelength band to a side of a second surface, and transmits the light of the first wavelength band in the second optical path to the side of the first surface and reflects the light of the second wavelength band in the second optical path to the side of the second surface; and
a band limiting unit that is disposed on the side of the first surface, transmits the light of the first wavelength band to the side of the first surface, and reflects the light of the second wavelength band to the side of the second surface.
   (15)

The medical observation system according to (14), in which the spectroscopic dispersion of the light in the first optical path and the spectroscopic dispersion of the light in the second optical path are performed by the same band limiting unit arranged on the spectral surface.

(16)

The medical observation system according to (15), in which
a first imaging element that images the light in the first optical path and a third imaging element that images the light in the second optical path are arranged on a third surface of the first optical member, and a second imaging element that images the light in the first optical path and a fourth imaging element that images the light in the second optical path are arranged on the second surface of the second optical member.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

1 Medical observation system
2 Insertion unit
3 Light source device
4 Light guide
5 Camera head
6 First transmission cable
7 Display device
8 Second transmission cable
9 Control device
10 Third transmission cable
21 Eyepiece unit
31 First light source
32 Second light source
50 Lens unit
52 Spectroscopic element
52a Spectroscopic element
52b Spectroscopic element
52c Spectroscopic element
54 Left-eye imaging unit
56 Right-eye imaging unit
58 Communication unit
91 Communication unit
92 First memory
93 Second memory
94 Observation image generation unit
95 Control unit
96 Input unit
97 Output unit
98 Storage unit
508a First region
512a Second region
520a First optical member
520b Second optical member
520c First surface
520d Second surface
520e Third surface
520f Fourth surface
540 First imaging element
542 Second imaging element
560 Third imaging element
562 Fourth imaging element
563 Dichroic mirror
514 Signal processing unit
521 Lens unit
522 Excitation light cut filter
523 Imaging element
523a Color filter
524 Signal processing unit
700 Base plate
700a Base plate
700b Base plate
700c Base plate
941 Memory controller
942 First image processing unit
943 Second image processing unit
944 Third image processing unit
945 Fourth image processing unit
946 Superimposed image generation unit
g10 to g26 Bonding portion
OP1 First optical path
OP2 Second optical path

1. A medical observation system, comprising:
a spectroscopic element that spectrally disperses light in a first optical path into light of a first wavelength band and light of a second wavelength band different from the first wavelength band, and spectrally disperses light in a second optical path different from the first optical path into light of the first wavelength band and light of the second wavelength band;
a first imaging element that images the light of the first wavelength band spectrally dispersed from the light in the first optical path;
a second imaging element that images the light of the second wavelength band spectrally dispersed from the light in the first optical path;
a third imaging element that images the light of the first wavelength band spectrally dispersed from the light in the second optical path; and
a fourth imaging element that images light of the second wavelength band spectrally dispersed from the light in the second optical path.

2. The medical observation system according to claim 1, wherein the spectroscopic dispersion of the light in the first optical path and the spectroscopic dispersion of the light in the second optical path are performed by the same spectroscopic element.

3. The medical observation system according to claim 2, wherein a spectral surface that spectrally disperses the light in the first optical path and a spectral surface that spectrally disperses the light in the second optical path are on the same plane of the same spectroscopic element.

4. The medical observation system according to claim 1, further comprising an image generation unit that generates a three-dimensional image of an output of the first imaging element, an output of the second imaging element, an output of the third imaging element, and an output of the fourth imaging element on a basis of the output of the first imaging element, the output of the second imaging element, the output of the third imaging element, and the output of the fourth imaging element.

5. The medical observation system according to claim 1, wherein the light in the first wavelength band is white light, and the light in the second wavelength band is first fluorescence.

6. The medical observation system according to claim 5, wherein when second fluorescence having a wavelength band different from that of the first fluorescence is imaged, the first imaging element and the third imaging element image white light and the second fluorescence in a time division manner.

7. The medical observation system according to claim 1, wherein
the spectroscopic element has a first region through which light imaged by at least one of the first imaging element or the second imaging element passes and a second region through which light imaged by at least one of the third imaging element or the fourth imaging element passes, and
in the spectroscopic element, a hole is provided in a region between the first region and the second region, and a convex portion of a base plate for fixing the spectroscopic element is fitted into the hole.

8. The medical observation system according to claim 7, wherein in the spectroscopic element, two of the holes are provided in the region between the first region and the second region, and two of the convex portions of the base plate for fixing the spectroscopic element are fitted into the holes.

9. The medical observation system according to claim 8, wherein one of the two holes of the spectroscopic element is a long hole.

10. The medical observation system according to claim 1, wherein
the spectroscopic element has a first region through which light imaged by at least one of the first imaging element or the second imaging element passes and a second region through which light imaged by at least one of the third imaging element or the fourth imaging element passes, and
in the spectroscopic element, a convex portion is provided in a region between the first region and the second region, and the convex portion is fitted into a hole of a base plate.

11. The medical observation system according to claim 1, wherein in the spectroscopic element, two convex portions are provided in a region between a first region and a second region, and the convex portions are fitted into two holes of a base plate.

12. The medical observation system according to claim 1, wherein one of two holes of a base plate is a long hole.

13. The medical observation system according to claim 1, further comprising a transmission unit that directly or indirectly transmits an output of the first imaging element, an output of the second imaging element, an output of the third imaging element, and an output of the fourth imaging element to an image generation unit that generates a three-dimensional image on a basis of the output of the first imaging element, the output of the second imaging element, the output of the third imaging element, and the output of the fourth imaging element.

14. The medical observation system according to claim 1, wherein
the spectroscopic element includes:
a first optical member that is disposed on a side of a first surface and transmits the light of the first wavelength band in the first optical path and the second optical path;
a second optical member that transmits the light of the first wavelength band in the first optical path to a side of the first surface that is a spectral surface and reflects the light of the second wavelength band to a side of a second surface, and transmits the light of the first wavelength band in the second optical path to the side of the first surface and reflects the light of the second wavelength band in the second optical path to the side of the second surface; and
a band limiting unit that is disposed on the side of the first surface, transmits the light of the first wavelength band to the side of the first surface, and reflects the light of the second wavelength band to the side of the second surface.

15. The medical observation system according to claim 14, wherein the spectroscopic dispersion of the light in the first optical path and the spectroscopic dispersion of the light in the second optical path are performed by the same band limiting unit arranged on the spectral surface.

16. The medical observation system according to claim 15, wherein
a first imaging element that images the light in the first optical path and a third imaging element that images the light in the second optical path are arranged on a third surface of the first optical member, and
a second imaging element that images the light in the first optical path and a fourth imaging element that images the light in the second optical path are arranged on the second surface of the second optical member.
